(19) United States
(12) Patent Application Publication
(10) Pub. No.: US 2002/0183709 A1
(43) Pub. Date: Dec. 5, 2002

(54) Hygiene product and process and apparatus for producing the same

(76) Inventor: Klaus Munsch, Neuwied (DE)

Correspondence Address: FASSE PATENT ATTORNEYS, P.A., P.O. BOX 726, HAMPDEN, ME, 04444-0726, US

(21) Appl. No.: 10/163,629

(22) Filed: Jun. 5, 2002

(30) Foreign Application Priority Data
Jun. 5, 2001 (DE) 101 27 278.2

(51) Int. Cl. A61F 13/15; A61F 13/20

(52) U.S. Cl. 604/385.05; 604/385.02; 604/385.201; 604/387

(57) ABSTRACT

The invention relates to a process and to an apparatus for producing hygiene products, in particular multi-layered sanitary towels each having an absorbent-material core and cover layers on both sides, the hygiene products first of all being produced just as initial products and then being provided with adhesive with the aid of a sheet material having at least one silicone separating layer, whereupon the hygiene products are packed individually with the aid of the same sheet material and/or are first of all folded, interconnected by virtue of the sheet material being stamped, and packed individually.

The essence of the invention is that first of all adhesive is applied to a sheet material, provided with a silicone separating layer, partially in the transporting direction or in the longitudinal direction of the sheet material, and that the sheet material and the initial products are then fed to one another, likewise in the longitudinal direction, and positioned one upon the other, and that the hygiene products are then folded together with the sheet material and packed individually in the sheet material.

### DESCRIPTION

The invention relates to a hygiene product and to a process and to an apparatus for producing the same.

In particular for the production of multi-layered sanitary towels each having an absorbent-material core and cover layers on both sides, it is known first of all to produce basic or initial products, that is to say first of all to provide products which still require at least one further process step in order to be completed. For this purpose, the initial products are additionally provided partially with adhesive with the aid of a sheet material having at least one silicone separating layer. The hygiene products completed in this way are then packed individually in the sheet material, which also serves as a substrate and for applying the adhesive. It is also known for the hygiene products to be folded before being packed and/or first of all for the sheet material to be fixed specifically by stamping, whereupon the stamping locations are severed in order for the hygiene products to be separated.

In the case of this prior art, the silicone separating layer is applied to web-like plastic sheet material in each case as a transversely extending strip, and the adhesive or the strips of glue are also located transversely on the sheet-material web or on the silicone strip. The sheet material is brought together with the almost completed hygiene product here such that the initial product is fed in a transversely located position to the plastic sheet material, moving in its longitudinal direction. As soon as the adhesive comes into contact with the initial product and adheres thereto, the hygiene product is complete, it being wrapped in the sheet material in a folding channel and folded at the same time. The folding lines of the hygiene product here are located in the movement direction of the still web-like sheet material. At the same time, the sheet material forms a continuous tube. Between two hygiene products provided with adhesive in each case, the tube is then closed by means of stamping lines and, finally, severed in the region of the stamping lines. The hygiene products are then in an individually packed state.

It is not only the case that this type of production requires the almost completed initial products to be transferred from a position in the longitudinal direction of the production line into a transverse position, but it is also necessary for the adhesive to be applied very precisely to the sheet material, and for the parts to be brought together absolutely precisely, that is to say in-register, in relation to one another. Otherwise the adhesive could not be transferred precisely to the predetermined locations on the initial products. Alternating speeds and spacings between the initial products as the latter are moved in the longitudinal direction, and then in the transverse direction, and the interaction with the fed sheet material, already partially provided with adhesive, make the production considerably more difficult. It would alternatively be possible for the sheet material just to be provided with silicone over the entire surface area. However, this would then result in it being difficult to stamp the sheet-material borders.

The object of the invention is thus to provide measures for simplifying to a significant extent the production process and, in particular, the application of the adhesive to one of the cover layers of the core as well as the packing of individual products. Furthermore, the intention is for this also to result in simplifications to the apparatus used for producing the products and, finally, in expedient improvements to the product itself.

In order to achieve this object, the invention provides that first of all adhesive is applied to a web-like sheet material, provided with a silicone separating layer, partially in the longitudinal direction, and that the sheet material and the initial products are then fed to one another, likewise in the longitudinal direction, and positioned one upon the other, and that, finally, the completed hygiene products, as they move further in the longitudinal direction, are packed in the sheet material and/or folded beforehand.

The production flow, on account of said process steps, is considerably more straightforward, and involves less outlay, than has been the case hitherto. In particular, however, an imprecise position of the parts in relation to one another has a less disruptive effect when they come into contact with one another. This applies, in particular, when, in the case of this process step being used according to the invention, a specifically provided, offset arrangement of the sheet material and product in relation to one another results in a novel possible production method and in a higher-grade product than has been the case hitherto.

It is also the case that the production speed can be increased in relation to the prior-art operation, this resulting in advantageous standardized production in the longitudinal direction of the parts in relation to production in the longitudinal direction and in the transverse direction, as has been the case hitherto.

Further features of the invention can be gathered from the subclaims and from the description in conjunction with the drawing.

The invention is described in more detail herebelow with reference to exemplary embodiments which are illustrated in the drawing, in which:

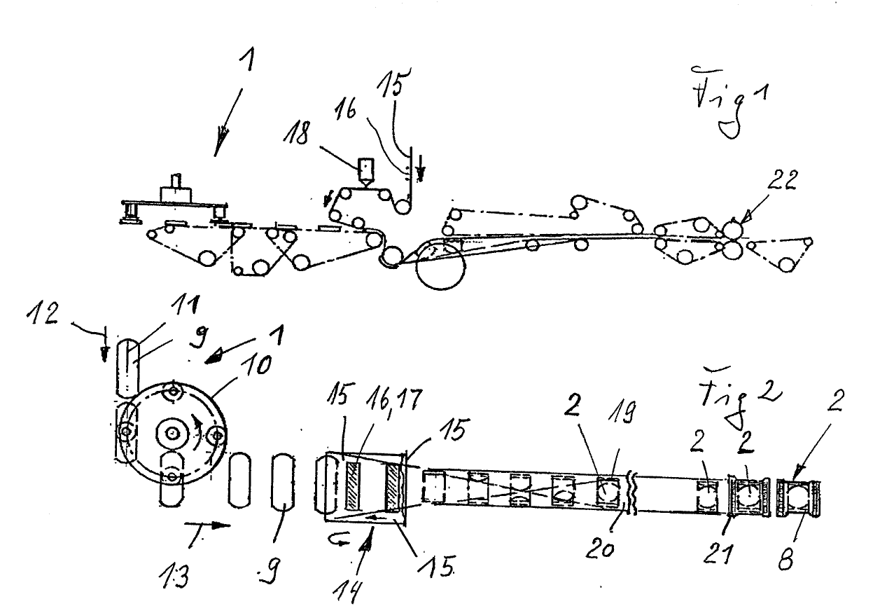

FIG. 1 shows, in a side view, a schematic diagram of an apparatus belonging to the prior art;

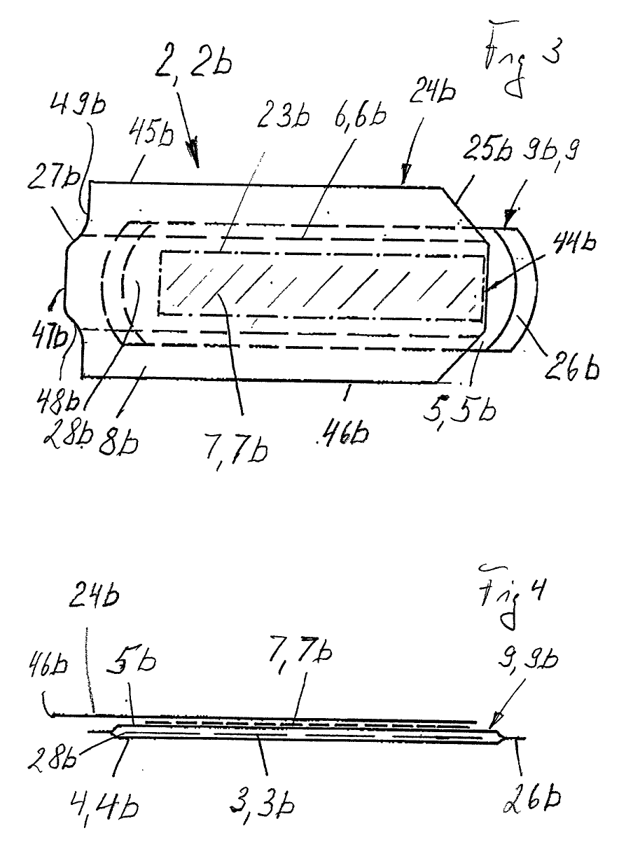

FIG. 2 shows a plan view of the apparatus according to FIG. 1;

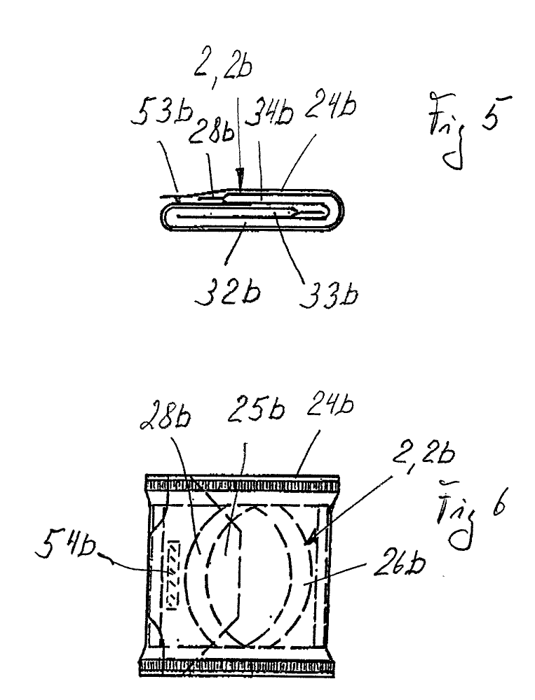

FIG. 3 shows a plan view of a hygiene product/sanitary towel with an associated piece of sheet material according to a preferred embodiment of the invention;

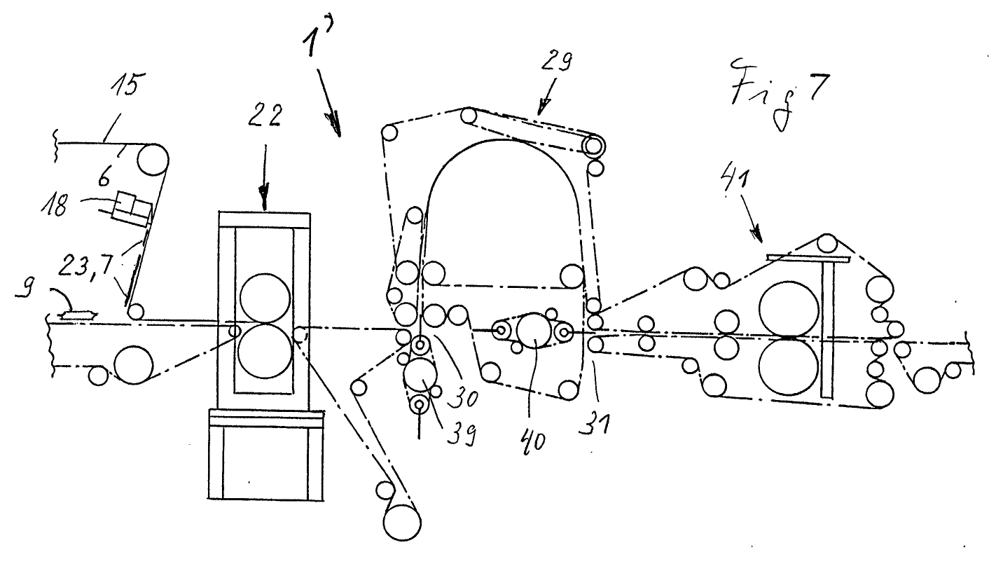

FIG. 4 shows, schematically, a side view of the parts according to FIG. 3;

FIG. 5 shows, schematically, a side view of an individually packed hygiene product according to FIG. 3;

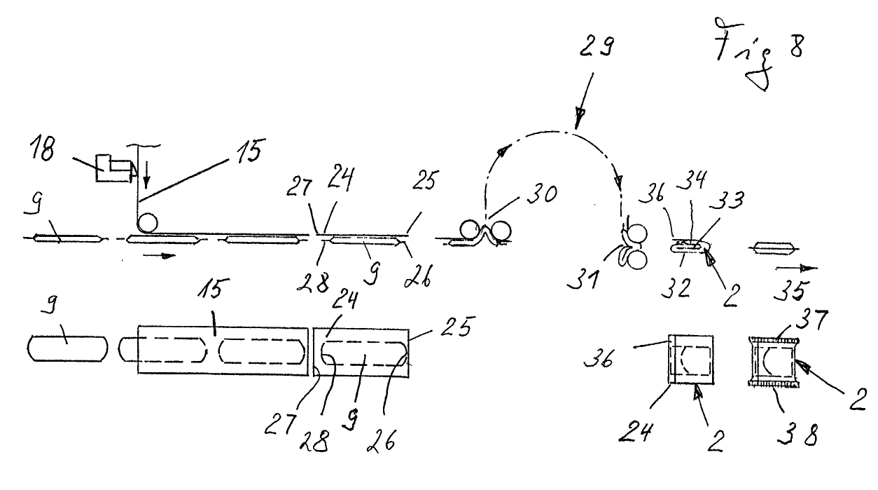

FIG. 6 shows a plan view of the individually packed hygiene product according to FIG. 3;

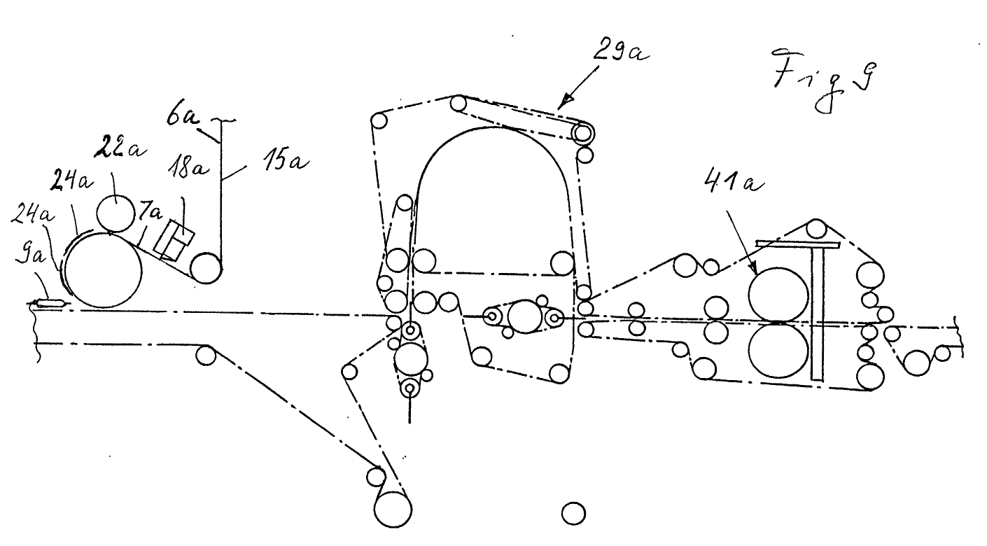

FIG. 7 shows, in side view, a schematic illustration of the apparatus for applying adhesive to an initial product and for packing an individually packed end product, that is to say the completed hygiene product, in a sheet material according to a first exemplary embodiment;

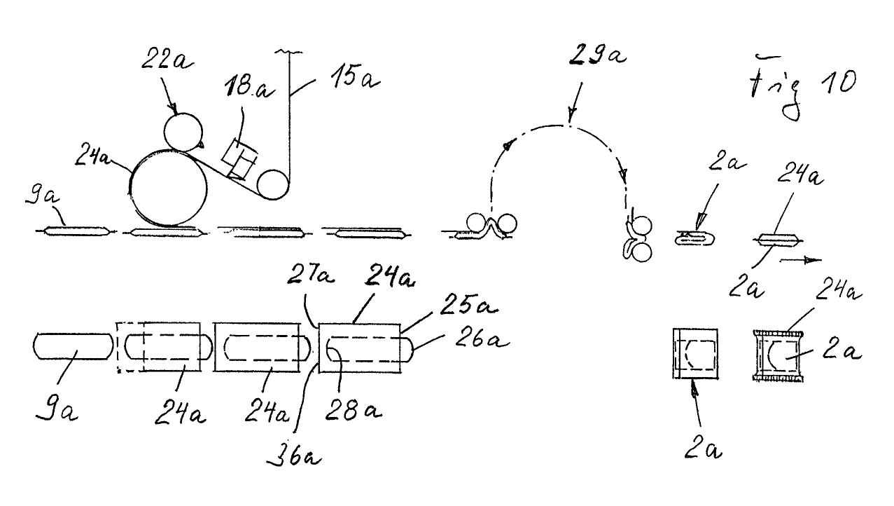

FIG. 8 shows, in each case schematically in side view and in plan view, the process steps for the apparatus according to FIG. 7;

FIG. 9 shows a view as in FIG. 7 of a modified embodiment; and

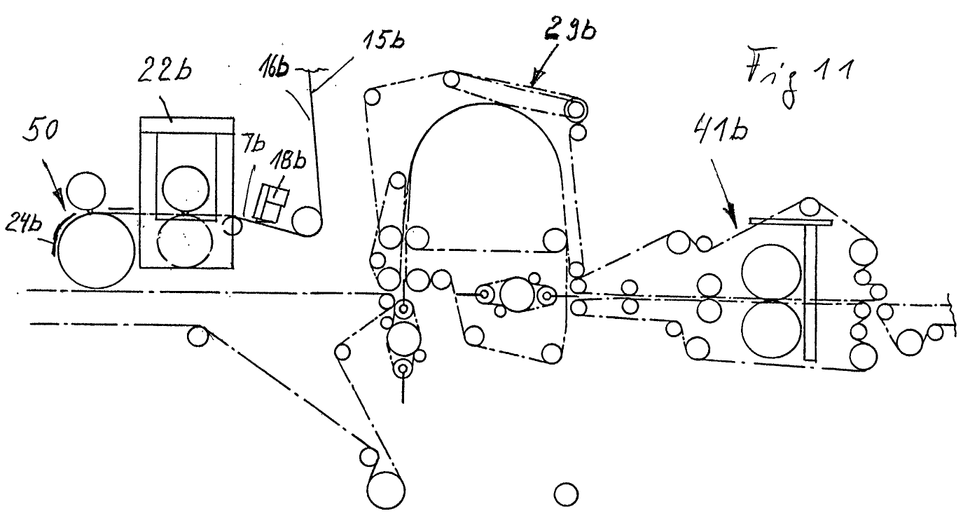

FIG. 10 shows, in each case schematically in side view and in plan view, the process steps for the apparatus according to FIG. 9;

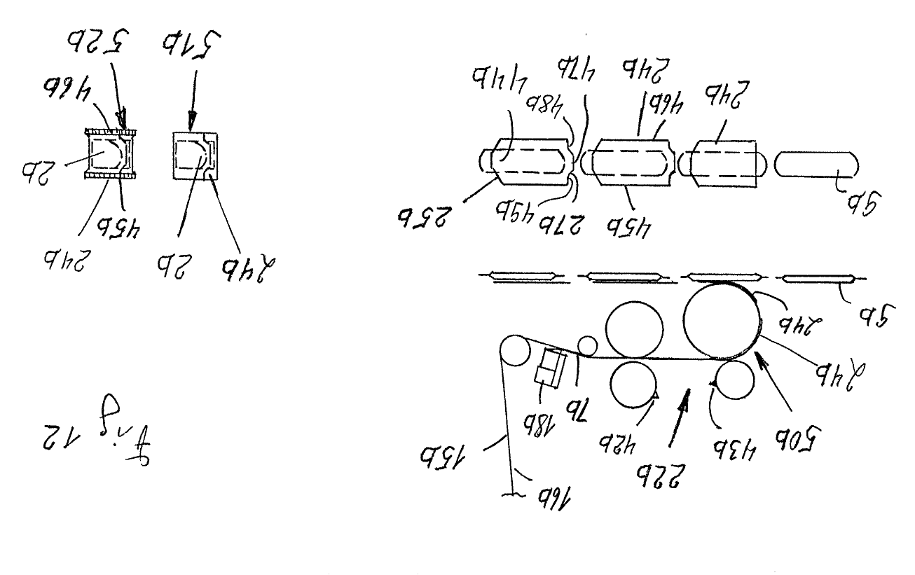

FIG. 11 shows a view as in FIGS. 7 and 9 of a further exemplary embodiment;

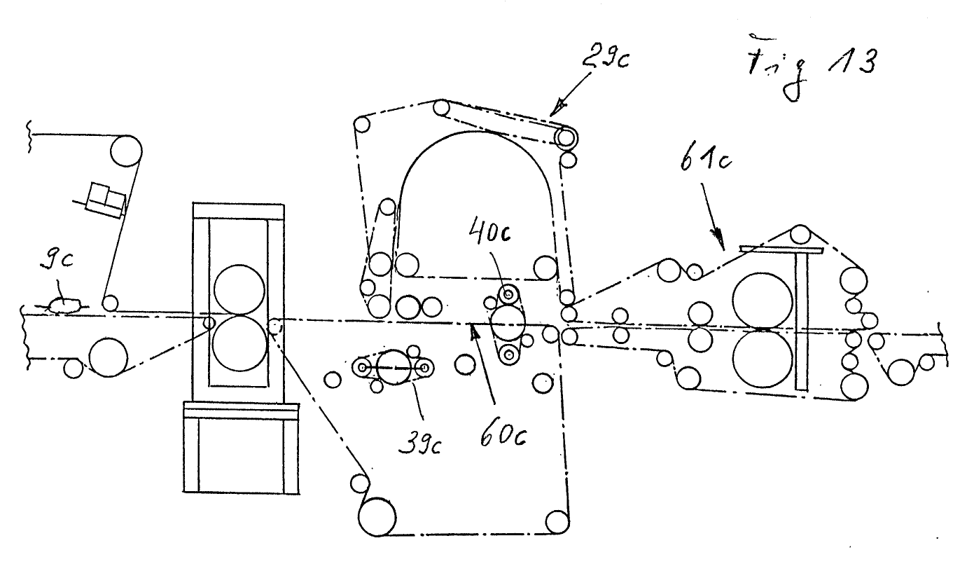

FIG. 12 shows, in each case only in part, a side view, plan view and schematic view of the process steps for the apparatus according to FIG. 11; and

FIG. 13 shows a view as in FIGS. 7, 9 and 11 of a final exemplary embodiment of the apparatus.

A known apparatus 1 for producing hygiene products 2 (for example sanitary towels), which—as in the case of the illustration in FIGS. 3 and 4—each have a core 3 and cover layers 4 and 5 on both sides and, on one of the cover layer [sic] 5, an adhesive layer 7 and are packed individually in a sheet material 8, comprises an arrangement (not illustrated in FIGS. 1 and 2) which produces the core 3, provided with the two cover layers 4 and 5, as the initial product 9 and, according to FIGS. 1 and 2, feeds the latter to a turntable 10. The initial products 9, which are received by the turntable 10 with their longitudinal direction or longitudinal axis 11 in the transporting direction according to the arrow 12, leave the turntable 10 following a quarter-turn and are then fed to a station 14 in a transversely located position, according to the prior art, corresponding to the arrow 13. In the station 14, a sheet material in the form of a sheet-material web 15 is positioned on the initial products 9. The sheet-material web 15 has a partial arrangement of transversely located silicone strips 16 as silicone separating layer 6 and bears, on the silicone strips 16, the adhesive 7, likewise extending transversely to the longitudinal direction of the sheet-material web 15 in each case, in the form of adhesive strips 17. These adhesive strips 17 have to be applied, by an adhesive-application arrangement 18, in a precisely timed manner to the silicone strips 16.

Once the sheet-material web 15 has been applied, in the station 14, to the in this case transversely fed initial products 9, the initial products 9, together with the sheet-material web 15 resting thereon, are folded to form a pack 19 with at least two layers, preferably three layers, and are wrapped in the sheet-material web 15 at the same time. In this case, the sheet-material web 15 forms a tube 20 which encloses the hygiene product 2. Upstream and downstream of the hygiene product 2 as seen in the transporting direction, the tube 20 is then stamped and thus closed upstream and downstream of the hygiene product 2. In the region of the stamping locations 21, the tube 20 is then severed with the aid of a severing apparatus 22, whereupon the hygiene product 2, according to the prior art, is in an individually packed state (FIG. 2).

An apparatus 1′ according to the invention (FIG. 7) for producing hygiene products 2 from initial products 9 comprises, for the purpose of applying adhesive 7 to the initial product 9, an adhesive-application arrangement 18, which likewise first of all partially applies the adhesive 7 to a silicone separating layer 6 on the sheet-material web 15. The silicone separating layer 6 is located as a narrow longitudinal strip on the sheet-material web and can even be applied to the sheet-material web by the manufacturer of the latter. Just as the silicone strip extends in the longitudinal direction of the sheet-material web 15, this also applies to the adhesive 7 applied by the adhesive-application arrangement 18, although, rather than being applied to the sheet-material web 15 as a continuous strip, the adhesive is applied in each case in the form of strip sections 23, which are shorter than the initial products 9 and than the completed hygiene initial [sic] product 2 (FIGS. 3 and 8).

The sheet-material web 15 provided with adhesive 7 is then, according to FIG. 7, positioned on the initial product or products 9 and, together with the latter, fed to a severing apparatus 22. There, the sheet-material web 15 is severed, with the result that pieces 24 of sheet material are located on the initial products 9 in each case (FIG. 8). The severing cut may be executed here such that the front end 25 of the piece of sheet material, as seen in the transporting direction, projects to a slight extent beyond the front end 26 of the product, while the rear end 27 of the piece of sheet material projects to a considerable extent beyond the rear end 28 of the product (FIG. 8). In this position in relation to one another, the initial product 9 and the piece 24 of sheet material pass into a folding apparatus 29, which, both in FIG. 7 and in FIG. 8, is only schematically illustrated. According to the exemplary embodiment, the initial product 9, together with the piece 24 of sheet material, is folded at two locations 30 and 31, that is to say twice, with the result that said initial product leaves the folding apparatus 29 with three layers 32, 33 and 34 located one upon the other. Moreover, the hygiene product 2, at the same time, is wrapped in the piece 24 of sheet material, of which the rear end 27, as seen in the transporting or running direction according to the arrow 35, projects, for example as an opening flap, some way beyond the end of the uppermost layer 34. At this process stage, the only thing which the piece 24 of sheet material, serving at the same time for packing purposes, still requires is to be secured against opening, for which purpose the piece 24 of sheet material, finally, is also stamped in the region of the outer side borders 37 and 38, as seen in transporting direction 35. The hygiene product 2 is thus individually and reliably fixed and packed in the piece 24 of sheet material.

In order to produce a three-layered product, the folding apparatus 29 comprises two push-rod arrangements 39 and 40, as can be seen from FIG. 7.

Also provided are a plurality of transporting arrangements and guide rollers, which are of no specific concern here. Basically the same applies to the apparatus 41, in which the side borders 37 and 38 of the folded product packed in the piece 24 of sheet material are closed by stamping. Both the folding apparatus 29 and the stamping apparatus 41 belong to the prior art and do not form part of the subject matter of the invention.

It is not absolutely necessary for the sheet-material web 15 to be severed once the sheet-material web 15 has been positioned on the initial products 9. According to a modification of the process according to the invention, it is also possible for an already separated piece 24a of sheet material to be positioned on an initial product 9a, as can be seen from FIG. 9 in conjunction with FIG. 10.

It is the case for this exemplary embodiment, and for all following exemplary embodiments, that basically equivalent parts in each case have the same designation and, in addition, an index letter.

According to the exemplary embodiment illustrated in FIGS. 9 and 10, the sheet-material web 15a is already subdivided into individual pieces 24a of sheet material, with the aid of a severing apparatus 22a, immediately downstream of the adhesive-application arrangement 18a. The piece 24a of sheet material, provided with a silicone separating layer 6a and adhesive 7a, is then positioned on the initial product 9a and, as in the case of the first-described process, the parts are then fed together to the folding apparatus 29a and the apparatus 41a for stamping and/or for closing the pieces 24a of sheet material, now serving as packaging.

The process by which already separated pieces 24a of sheet material are positioned on the initial product 9a is advantageous, in particular, because it is possible for the pieces 24a of sheet material to be positioned in an offset manner on the initial products 9a (see plan view in FIG. 10). This expediently takes place, according to the product example illustrated in FIG. 10, such that the front end 26a of the product projects to a considerable extent beyond the front end 25a of the piece of sheet material, while the rear end 27a of the piece of sheet material projects beyond the rear end 28a of the product. The rear end 24a [sic] of the piece of sheet material may thus, in turn, serve as a tear-open flap, as is also the case for the abovedescribed exemplary embodiment.

According to a further exemplary embodiment, which is illustrated in FIGS. 11 and 12, the sheet-material web 15b provided with a silicone strip 16b, is likewise expediently subdivided into individual pieces 24b of sheet material once adhesive 7b has been applied with the aid of an adhesive-application arrangement 18b. For this purpose, use is made of a severing apparatus 22b, although, rather than just producing rectilinear cuts in the direction transverse to the running direction of the sheet-material web 15b, as in the case of the exemplary embodiment illustrated in FIGS. 9 and 10, this severing apparatus produces shaped cuts or contoured cuts or contoured cuts and severing cuts at or for the two ends of the pieces 24b of sheet material. For this purpose, the severing apparatus 22b, according to the exemplary embodiment which is likewise only schematically illustrated in FIGS. 11 and 12, has two cutters 42b and 43b, which act at a distance apart from one another.

The shape and form of the cutters 42b and 43 [lacuna] preferably differ such that the front cutter 42b as seen in the running direction, executes lateral contoured cuts in the sheet-material web 15b and the other cutter 43b executes severing cuts for the purpose of separating the pieces 24b of sheet material.

According to the exemplary embodiment illustrated in FIGS. 3 and 12, the contour at the two ends 25b and 27b of the piece of sheet material is expediently selected such that the central region 44b, as seen in the transporting direction or longitudinal direction, of the piece 24b of sheet material projects in the running direction, and/or counter to the transporting direction, beyond at least one of the lateral borders 45b and 46b at at least one end. According to the exemplary embodiment, the lateral borders 45b and 46b are thus set back in relation to the central region 44b or are shorter than the central region 44b of the piece 24b of sheet material.

According to the exemplary embodiment, the front end 25b of the piece of sheet material, as seen in the transporting direction, during production is trapezoidal. The rear end 27b of the piece of sheet material has a rectilinear, central border section 47b and, alongside this, in each case arcuate border sections 48b and 49b, which run into the lateral borders 45b and 46b.

Once the piece 24b of sheet material has been positioned on the initial product 9b in the station 50b, the adhesive 7b adheres to the initial product 9b, with the result that the hygiene product 2 is complete. The hygiene product 2 then passes into the folding station 29b and there, according to the exemplary embodiment, is folded into three layers 32b, 33b and 34b located one upon the other and, at the same time, is wrapped in the piece 24b of sheet material. The hygiene product 2b thus leaves the folding apparatus 29b as a semi-packed product 51b and then passes into the apparatus 41b (FIG. 11) for stamping the lateral borders 45b and 46b of the piece 24b of sheet material.

The hygiene product 2b leaves the apparatus 41b as a fully and individually packed end product 52b .

The arrangement of the individual layers 32b, 33b and 34b can also be seen, in particular, from FIG. 5, it being possible for the rear, projecting border 46b of the piece 24b of the sheet material to be fixed, as a tear-open flap 53b, on the central layer by an area 54b of adhesive (FIGS. 5 and 6).

In the packed state according to FIG. 6, the front end 25b of the piece of sheet material, as seen in the transporting direction, during production is located in an offset manner in relation to the front end 26b of the product. According to FIG. 5, the rear end 28b of the product forms the top layer 34b in the packed state.

It is not absolutely necessary, in principle, for the hygiene products 2, 2b to be packed in the folded state. It is also possible, in principle, for them to be packed individually, or in groups, in a non-folded state. For this reason, a bypass section 60c is provided in the region of the folding apparatus 29c, or for bypassing the folding apparatus 29c, with the result that non-folded products can be fed directly to a packaging machine.

The product already forms a completed product in the region of the bypass section.

The bypass section 60c expediently extends through the folding station 29c in rectilinear continuation of the upstream apparatus. In order that the bypass section 60c can lead rectilinearly through the folding station 29c, the push-rod arrangements 29c and 40c are arranged in a pivotable manner, with the result that, in bypass operation, they can be moved out of the path over which the products move to the stamping station 61c (FIG. 13).

Finally, it is expedient the [sic] a strip of silicone paper to be used instead of a sheet material if the products are transported through the bypass section.

It goes without saying, finally, that, rather than the invention being restricted to the exemplary embodiments illustrated in the figures, it is also possible to have modifications without departing from the basic idea of the invention.

The essential factor, finally, is that the products, namely absorbent articles, are individually packed, the central separating strip and tab separating parts being fastened permanently on the packaging.

Furthermore, the packaging has an adhesive part at one end. It is also possible for the packaging to have a lateral adhesive strip at one end.

In principle, the longitudinal side borders of the packaging are connected to one another such that they can be torn open.

The folding axes of the packaging are aligned in relation to the longitudinal borders of the absorbent insert.

The invention thus basically deals with a process for packing individual, absorbent inserts which has [sic] two longitudinal borders and two side borders as well as a liquid-permeable, front surface element, a liquid-impermeable, rear surface element and an absorbent core, which is inserted between the two surface elements.

The invention also deals with providing packaging for a part in a generally flat position, and with fastening a central separating strip on the packaging such that the longitudinal axis of said strip generally forms a right angle in relation to the longitudinal axis of the packaging, and with fastening a pair of tab separating parts on the packaging on each side of the central separating strip.

Instead of a sheet material, of a sheet-material web or even of a strip of silicone paper, it is basically also possible to use any other desired material that is similarly suitable for packing the products. Using a sheet material, in particular a plastic sheet material, is thus just one possible way of implementing the invention.

1. Process for producing hygiene products (2, 2b), in particular multi-layered sanitary towels each having an absorbent-material core (3) and cover layers (4, 5) on both sides, the hygiene products (2, 2b) first of all being produced just as initial products (9, 9b) and then being provided with adhesive (7, 7b) with the aid of a sheet material having at least one silicone separating layer (6, 6b), whereupon the hygiene products (2, 2b) are packed individually with the aid of the same sheet material and/or are first of all folded, interconnected by virtue of the sheet material being stamped, and packed individually, characterized
a) in that first of all adhesive (7, 7b) is applied to a sheet material, provided with a silicone separating layer (6, 6b), partially in the transporting direction or in the longitudinal direction of the sheet material,
b) in that the sheet material and the initial products (9, 9b) are then fed to one another, likewise in the longitudinal direction, and positioned one upon the other, and
c) in that the hygiene products (2, 2b) are then folded together with the sheet material and packed individually in the sheet material.

2. Process according to claim 1, characterized in that the sheet material is positioned on the initial product (9) in the form of a sheet-material web (15), which bears the adhesive (7), and only then is severed.

3. Process according to claim 1, characterized in that the sheet-material web is cut into pieces (24a, 24b) of sheet material before being positioned on the initial product (9a, 9b), and is positioned on the initial product (9a, 9b) as the adhesive-bearing piece (24a, 24b) of sheet material.

4. Process according to claim 1, characterized in that the piece (24a, 24b) of sheet material is positioned on the initial product (9a, 9b) in an offset manner in the longitudinal direction.

5. Process according to claim 1, characterized in that, when severed from the sheet-material web (15b), the piece (24b) of sheet material is provided with a contour differing from a rectilinear cut at the front end (25b) of the piece of sheet material and/or at the rear end (27b) of the piece of sheet material, as seen in the longitudinal direction.

6. Process according to claim 1, characterized in that, when severed from the sheet-material web (15b), the piece (24b) of sheet material is provided with a contour such that the central region (44b), as seen in the transporting direction/longitudinal direction, of the piece (24b) of sheet material projects in the running direction, and/or counter to the transporting direction, beyond at least one of the lateral borders (45b, 46b) at at least one end.

7. Process according to claim 1, characterized in that the piece (24a, 24b) of sheet material is positioned in an offset manner on the initial product (9a, 9b) such that the front end (26a, 26b) of the product, as seen in the production direction, at least partially projects beyond the front end (25a, 25b) of the piece of sheet material, and in that the rear end (27a, 27b) of the piece of sheet material at least partially projects beyond the rear end (28a) of the product, counter to the transporting direction.

8. Process according to claim 1, characterized in that the initial product is folded together with the piece of sheet material, in a folding apparatus, to form a product with two or more layers, in particular three layers.

9. Process according to claim 1, characterized in that the hygiene product (2b), following the folding operation, using a projecting end (27b) of the piece of sheet material and with the aid of areas (54b) of adhesive, is fixed/closed in the piece (24b) of sheet material serving, at the same time, as packaging.

10. Process according to claim 1, characterized in that at least one end (25b) of the piece of sheet material is contoured, and is preferably contoured trapezoidally.

11. Process according to claim 1, characterized in that the front end (25b) of the piece of sheet material, as seen in the production direction, is trapezoidal.

12. Process according to claim 1, characterized in that one end (27b) of the piece of sheet material has a rectilinear, central border section (47b) and, adjoining this on the outside in each case, arcuate border sections (48b, 49b), which run into lateral borders (45b, 46b) of the piece (24b) of sheet material.

13. Process according to claim 1, characterized in that, in the case of a bypass section, a strip of silicone paper is used instead of a sheet material.

14. Hygiene product, in particular in the form of a multi-layered sanitary towel which in each case has an absorbent-material core (3) and cover layers (4, 5) on both sides, is produced as an initial product (9, 9a, 9b) and then is provided with adhesive (7, 7a, 7b) with the aid of a sheet material having at least one silicone separating layer (6, 6a, 6b), and is packed in the same sheet material and/or is first of all folded in layers (32, 33 and 34) and then packed individually, characterized in that the silicone separating layer (6, 6a, 6b) extends in the longitudinal direction on the sheet material and is narrower than the sheet material, and in that the adhesive (7, 7a, 7b) is arranged partially on the silicone separating layer (6, 6a, 6b).

15. Hygiene product according to claim 14, characterized in that the sheet material is arranged, as a piece (24a, 24b) of sheet material, in an offset manner on the initial product (9a, 9b).

16. Hygiene product according to claim 14, characterized in that at least one end (27a, 27b) of the piece of sheet material projects beyond at least one end (28a, 28b) of the product.

17. Hygiene product according to claim 14, characterized in that one end (26a, 26b) of the product projects beyond the associated end (25a, 25b) of the piece of sheet material, and in that the other end (27a, 27b) of the piece of sheet material projects beyond the other end (28a, 28b) of the product.

18. Hygiene product according to claim 14, characterized in that at least one end of the piece of sheet material has a non-rectilinear contour.

19. Hygiene product according to claim 14, characterized in that at least one end of the piece of sheet material is trapezoidal.

20. Hygiene product according to claim 14, characterized in that one end (27b) of the piece of sheet material has a rectilinear, central border section (47b) and, adjoining this on the outside in each case, arcuate border sections (48b, 49b), which run into lateral borders (45b, 46b) of the piece (24b) of sheet material.

21. Hygiene product according to claim 14, characterized in that the lateral borders (45b, 46b) of the piece (24b) of sheet material are stamped for packing purposes.

22. Hygiene product according to claim 14, characterized by an arrangement with two or more layers, in particular three layers, in the piece of sheet material serving as packaging.
